SEQUENCE LISTING (6 sequences)

SEQ ID NO: 1 - RNA, 22 nt, Pseudomonas putida
  misc_feature (1)...(22): 16SrRNA
gaacugaagaguuugaucaugg

SEQ ID NO: 2 - RNA, 22 nt, Rhodococcus equi
  misc_feature (1)...(22): 23SrRNA
auuuacugucugagcugggugg

SEQ ID NO: 3 - RNA, 22 nt, Rhodococcus equi
  misc_feature (1)...(22): 23SrRNA
caggcguagccgauggacaacg

SEQ ID NO: 4 - RNA, 22 nt, Rhodococcus equi
  misc_feature (1)...(22): 23SrRNA
cgaauagggcgaucguaguggc

SEQ ID NO: 5 - RNA, 23 nt, Culex pipiens
  misc_feature (1)...(23): miR-263a
aauggcacuggaagaauucacgg

SEQ ID NO: 6 - RNA, 23 nt, Musca domestica
  misc_feature (1)...(23): Bantam
ugagaucauugugaaagcugauu

(19) United States
(12) Patent Application Publication
(10) Pub. No.: US 2014/0005054 A1
(43) Pub. Date: Jan. 2, 2014

(54) COMPLEX RNA COMPOSITION OF BODILY FLUIDS

(71) Applicant: INSTITUTE FOR SYSTEMS BIOLOGY, SEATTLE, WA (US)

(72) Inventors: David GALAS, Seattle, WA (US); Kai WANG, Bellevue, WA (US); Paul WILMES, Bettembourg (LU)

(73) Assignee: INSTITUTE FOR SYSTEMS BIOLOGY, Seattle, WA (US)

(21) Appl. No.: 13/916,461

(22) Filed: Jun. 12, 2013

Related U.S. Application Data
(60) Provisional application No. 61/658,876, filed on Jun. 12, 2012.

(51) Int. Cl. C12Q 1/68

(52) U.S. Cl. CPC C12Q 1/6895; C12Q 1/6888; C12Q 1/689
USPC 506/2; 506/16

(57) ABSTRACT

Determination of the level and identity of various RNA molecules in circulation or tissues of subjects permits determination of microbiome composition and function of the subject as well as providing an index to health of various organs. Because rapid parallel sequencing techniques are available, such determinations can be conducted on a practical level.

### CROSS REFERENCE TO RELATED APPLICATION

This application claims priority from U.S. Ser. No. 61/658,876 filed 12 Jun. 2012. The content of this document is incorporated herein by reference.

### TECHNICAL FIELD

The invention relates to assessing the character and level of RNA molecules in human tissues and bodily fluids especially plasma. In particular, it relates to the nature and level of a multitude of both endogenous and exogenous RNA in these samples, including determining microbiome composition and function for a test subject.

### BACKGROUND ART

Many biological insights have emerged from the analysis of DNA and RNA sequences. Important discoveries, such as various pathology-causing variants in the human genome and the history of human migration, were made possible by the availability of sequencing technology. Normal human physiology is the result of a well-orchestrated balance between genetic (intrinsic) and environmental (extrinsic) factors, and the availability of the complete human genome sequence facilitates the study of complex human-environmental interactions. Recently this has included the human-microbiome interaction, especially the gut microbiome. These microbes interact intimately with gut epithelium and the alteration in the spectrum of the gut microbiome has been linked to various physiopathological conditions, such as diarrhea, diabetes, obesity, inflammatory pathologies and cancer as well as to the general state of health.

The recent development of highly parallelized next generation (NextGen) sequencing technologies has further advanced the use of sequencing as a tool for studying complex biological systems by genome sequencing and transcriptome analysis. One advantage of using a sequence-based approach for transcriptome analysis is the ability to identify novel transcripts, such as alternative usage of exons or polyadenylation sites of known transcripts. The recent explosion of information on microRNA (miRNA) and other noncoding RNAs (ncRNAs) is the result in part of applying these new technologies. To date more than 1000 different human miRNA species have been identified (see miRBase, see the website for mirbase.org). Recently, a significant number of these RNA molecules have been observed in the extracellular environment and have been implicated as important mediators in cell-cell communication.

### DISCLOSURE OF THE INVENTION

The present invention relates to the application of RNA identification techniques such as parallel rapid sequencing and microarray mass spectrometry techniques to identify and quantify the RNA molecules circulating in blood, residing in tissues, or present in other bodily fluids. It has been found that not all of the circulating RNA molecules are endogenous to human or other animal subjects, and many are characteristic of exogenous substances or organisms, such as bacteria, archaea, fungi, or substances that have been consumed such as food or infectious organisms. These exogenous RNAs have also been observed in tissues. A variety of applications is disclosed as part of the invention.

Thus, in one aspect, the invention is directed to a method to assess the physiological state of a test subject which method comprises obtaining a test spectrum of the identity and level of RNA molecules present in a sample of a tissue or biological fluid from said test subject; and comparing said spectrum with a control spectrum comparably obtained from one or more normal, control subjects; whereby a significant difference between the test spectrum from that of said control spectrum indicates a physiological condition in said test subject that is other than normal.

In another aspect, the invention is directed to a method to determine microbiome composition and function of a test subject, which method comprises obtaining a test spectrum of the identity and level of RNA molecules present in a sample of a tissue or biological fluid from said test subject; and associating the identity and/or level of RNA molecules in said spectrum with individual microorganisms; whereby the microbiome of said subject is determined.

In still another aspect the invention is directed to a method to assess the effect of a treatment or protocol that has been administered to a test subject, which method comprises obtaining a test spectrum of the identity and level of RNA molecules present in a sample of a tissue or biological fluid from said test subject; and comparing said spectrum with a control spectrum comparably obtained from one or more subjects that have not been administered said treatment or protocol or from said subject prior to administration of said treatment or protocol; whereby a significant difference between the test spectrum from said control spectrum indicates the effect of said treatment or protocol on said test subject.

In still another aspect the invention is directed to a method to determine whether a test subject has been subjected to a treatment or protocol or is afflicted with a disease or condition, which method comprises obtaining a test spectrum of the identity and level of RNA molecules present in a sample of a tissue or biological fluid from said test subject; and comparing said spectrum with a control spectrum comparably obtained from one or more control subjects that have been administered said treatment or protocol or are known to be afflicted with said disease or condition; whereby a significant similarity between the test spectrum with that of said control spectrum indicates the subject has been administered said treatment or protocol or is afflicted with said disease or condition.

In still another aspect the invention is directed to a method to determine whether a subject has ingested one or more substances, which method comprises obtaining a test spectrum of the identity and level of RNA molecules present in a sample of a tissue or biological fluid from said subject; and comparing said test spectrum with a control spectrum comparably obtained from one or more subjects that have ingested said one or more substances, whereby a significant similarity between the test spectrum with that of said control spectrum indicates the subject has ingested said one or more substance.

In still another aspect the invention is directed to a method to determine whether a subject has ingested one or more substances which method comprises obtaining a test spectrum of the identity and level of RNA molecules present in a sample of a tissue or biological fluid from said subject; and associating the identity and/or level of RNA molecules in said spectrum with said one or more substances; whereby assessing the presence and/or level of one or more RNA molecules as characteristic of said one or more substances determines whether said ingestion has occurred. This general principle can be expanded to correlate dietary patterns with patterns found in the microbiome. Thus, combinatorial techniques can be used to correlate differences in dietary patterns with regard to single types of nutrients or multiplicities of types of nutrients with changes in the microbiome. This may guide practitioners in prescribing appropriate dietary changes for subjects.

In still another aspect the invention is directed to a method to identify a biological pathway that is affected in a subject afflicted with an abnormal condition, which method comprises identifying at least one RNA molecule in the RNA spectrum of a sample of tissue or biological fluid of said subject, the presence or level of which is different in from that in a control spectrum comparably obtained from control subjects; testing the effect of said RNA molecule on the transcriptome of cells of the same species as the test subject; identifying at least one element of said transcriptome that is affected; and associating said element with a biological pathway.

In addition to the methods of the invention, the information useful in conducting the methods can be tabulated and stored on computer-readable media. Thus, the invention further includes a database contained on a computer readable medium which comprises a record of the identity and levels of RNA contained in an RNA spectrum associated with at least one of: 1) tissue or biological fluid of normal subjects; 2) tissue or biological fluid of subjects affected by known conditions; 3) tissue or biological fluid of subjects or administered known treatments; 4) tissue or biological fluid of subjects known to have ingested specified substances.

The methods of the invention may be performed on human subjects or on any vertebrate subject, including laboratory animals as well as livestock, companion animals, horses, and the like.

### BRIEF DESCRIPTION OF THE DRAWINGS

FIG. 1 shows the schema of the sequence mapping protocol. A “map and remove” process was adapted to map reads against various sequence databases (left dotted box) in specific order as indicated. We allowed different levels of sequence mismatch tolerance, 0 mismatch, 1 mismatch and 2 mismatches only when comparing the sequence reads against human sequence database.

FIGS. 2A-2C show distribution of sequence reads from human plasma (A), other sample types (B) and public domain data (C) among different sequence categories. The sample identifies were listed on the top, the sequence mapping criteria were indicated on the bottom and the list of different sequence categories is indicated on the right of each figure.

FIGS. 3A-3G show distribution of sequence reads from human plasma mapped to bacteria, archaea (A to C) and fungi (D to F) phylum. The Y-axes are the numbers of reads in log 10 value and individual phyla are indicated on the X-axis. The number of reads used in the figures represents the average of all 9 plasma samples used in the study. The solid bars represent the total number of processed reads mapped to specific phyla while open bars are the number after removing rRNA and tRNA reads. The individual bacterial and fungal species with the most abundant processed reads (B and E) and processed reads after removing tRNA and rRNAs (C and F) are also shown. The bacteria and fungi RNA can also be detected directly in plasma from small blood samples from finger pricks (G). The results shown are the averages from 5 healthy donors. The identity of the sequence detected is provided on the X-axis and the level of RNA (in 40-Ct value) is indicated on the Y-axis.

FIGS. 4A-4C show number of sequence reads mapped to common food items such as cereal grains (A) and others (B). The Y-axes are the number of reads in log 10 value and individual species are indicated on the X-axis. The number of reads used in the figures represented the averages from all 9 plasma samples used in the study. FIG. 3C shows the difference in the abundance of reads mapped to common cereal gains between a Chinese individual (gray bars) and the (Caucasian) samples used in the study (solid bars).

FIG. 5 shows levels of albumin, apoA2 and transferrin RNA in plasma after treatment with acetaminophen.

FIG. 6 shows the relative changes of RNA concentrations after treating the plasma with DNase, RNase, Protease and Triton™ X-100. The plasma samples were treated with various conditions (indicated on the top of the figure) prior to RNA isolation. The Y-axis represents the relative concentration change compared to no treatment determined by qPCR. The data represents the average changes from 9 plasma samples. The black bars represent the changes of an endogenous miRNA, miR-16, the open bars are exogenous miRNA, miR-263 from mosquitos and the gray bars are the 16S rRNA from Pseudomonas putida.

FIG. 7 shows the structures of RNA used to transfect mouse cells for determination of effect on pathways.

FIG. 8 shows the results of expression levels of various genes corresponding to RNA of FIG. 7.

### MODES OF CARRYING OUT THE INVENTION

The present invention takes advantage of the availability of RNA identification techniques such as high throughput parallel sequencing techniques, such as the commercially available NextGen techniques as well as microarray/mass spectrometry techniques to explore the implications of the spectrum of RNA molecules found in bodily fluids and tissues. Although the examples herein focus on plasma, RNA profiles may also be obtained from other biological fluids such as saliva, semen, lymph, urine and in tissues themselves either as secretions or extracts. Depending on interest, the subjects may be laboratory models such as rabbits, mice, rats, guinea pigs, etc., or other animals such as livestock, birds, fish, as well as animals in general such as companion animals, racehorses and marsupials. A number of applications of such spectra are part of the present invention.

By “RNA spectrum” of a biological fluid or tissue we mean the identity and quantity or concentration of a multiplicity of RNA sequences or molecules present in the tissue or biological fluid. As shown in the examples below, tissues or fluids may contain not only RNA representing the transcriptome and miRNAs, but may also contain exogenous sequences characteristic of microorganisms, i.e., the microbiome represented in the fluid or tissue by its specific RNA spectral signature. Other exogenous RNAs may result from ingested materials such as plant materials or animals ingested as food as well as microbial contaminants of these ingested materials or other substances. Thus, the information obtained by determining the RNA spectrum may have forensic value to determine whether ingestion of materials having informative RNA patterns has occurred. Typically, the RNA sequences or molecules are 10-40 nucleotides in length, or may be 15-35 nucleotides in length or may be 20-25 nucleotides in length. All integer values between the designated ranges are included—thus, sequences or molecules of 10-35 nucleotides in length also include those 14-30 nucleotides in length, or 16-29 nucleotides in length, etc.

The identification of these RNA molecules or sequences is performed by matching these to publicly available or other databases that contain sequence information regarding the microRNA (miRNA), genetic sequences, or transcriptomes of the organism from which the tissue of biological fluid used to sample is derived and matching the RNA sequences or molecules in the spectrum to those in the database. The matching can be conducted using a number of strategies, for example, allowing no mismatches, or one mismatch or two mismatches to account for allelic variations, etc. Typically, microRNA sequences or molecules in the RNA spectrum are not permitted any mismatches because of the similarity of miRNA's, but RNA sequences or molecules that otherwise match the transcriptome or the genomic sequences of the organism may be allowed greater flexibility. This permits identification of molecules or sequences in the spectrum that cannot be matched endogenously to be more efficiently compared to other databases that represent the genomes, transcriptomes, or microRNA of microorganisms or substances such as food substances that might be present in a microbiome or other exogenous sequences in the organism tested.

The number of RNA molecules composing a determined RNA spectrum is arbitrary, but typically the spectrum will comprise more than one such RNA molecule. However, determination of the nature and quantity even of a single RNA is informative under some circumstances—e.g., an RNA specifically characteristic of anthrax would demonstrate ingestion of this microorganism. Typically, however, a multiplicity of RNA molecules is identified and optionally quantitated to obtain a specific “RNA spectrum” of a fluid or tissue derived from a subject. Thus, the number of RNA molecules to be characterized and optionally quantitated may be as few as two or as many as several hundred. All integer numbers between 2 and 100 are also included as if specifically set forth herein. Thus, the spectrum may contain, for example, 3, 5, 20, 50 or 100 such molecules; again, it is to be emphasized that any and all specific integers between these boundaries are to be considered specifically set forth herein.

The “microbiome” of a sample of tissue or fluid is an RNA spectrum that represents RNA associated with microorganisms and viruses. Microorganisms include fungi, bacteria, archaea and protozoa, and any single-celled or non-cellular microbe.

The sample size for determination may be quite small and is arbitrary and suited to the specific method for determination of the spectrum.

Many of the applications of the invention involve comparisons between test and control spectra. These spectra are “significantly similar” if statistical tests indicate that they vary overall by <10%, preferably <5% and preferably <1%. Conversely, they are “substantially different” if they differ by at least 1% overall, preferably 5% overall and more preferably 10% or more overall. In many cases, it is not necessary to apply statistics; a graphic display of a manageable number of RNA molecules in each spectrum may be sufficient for simple observation to determine whether the spectra are similar or different. Many algorithms are also available to determine statistical similarities and differences and any such algorithms may be applied to make this determination.

As noted above, the substances that may contribute to the RNA spectrum are ingested substances, and “ingestion” includes not only oral uptake, but any means of providing the substance to the subject, including injection, transmucosal delivery, transdermal delivery, and any mechanism that succeeds in providing the substance to the subject. Thus, the substance may be supplied, for example, to a tumor by direct administration to the tumor such as by injection, and may be provided in a multiplicity of forms. The examples below illustrate the effect of oral ingestion of foodstuffs, but the presence of insect RNA in plasma indicates that inhalation may also be a route of administration effective in delivering exogenous RNA. Any material capable of generating, or having associated with it, RNA is included within a “substance” to be ingested. “Substance” is not limited to single molecules but includes mixtures, composites, organisms, materials in general, including those containing contaminants.

By associating the identities of RNA molecules in the spectrum with their sources, is meant that by virtue of the nature of the sequence of the RNA, it can be determined to have originated in a particular source. Thus, if the RNA is characteristic of a particular substance or organism or microbe, its presence and/or quantity is informative as to the exposure of the subject to the substance or organism. Some, indeed many, RNA molecules are not uniquely characteristic of a particular source exogenous to the subject, but the level present in the fluid or tissue may indicate that the RNA present endogenously has been supplemented. Further, the substance itself may not contain or generate RNA but may stimulate alterations in the patterns of RNA of the subject. Thus, toxins, pharmaceuticals, and other inorganic or organic small molecules or non-living molecules in general by virtue of their perturbation of the metabolism and physiology of the subject will alter the RNA spectrum. This expands the applications for forensic purposes. For example, detection of a pattern characteristic of arsenic poisoning or ricin poisoning will indicate that such poisoning has or has not occurred.

The inventors have also found that the nature of the RNA spectrum is useful to determine metabolic and other physiological pathways that are associated with particular diseases or conditions. Thus, the nexus between the impact of particular RNA molecules on known pathways can be determined by measuring the effects of such RNAs on cells of the same species as the subject. For example, if the subject shows elevated levels of an RNA in plasma that is associated with enhancing a pathway associated with oncogenesis, the presence and amount of this RNA in the spectrum may indicate the relevance of this pathway to tumor progression, thus providing a target for treatment.

In still another embodiment, the invention takes advantage of the discovery by applicants that RNA molecules are protected in plasma and the circulatory system in general by association with protein and/or lipid complexes. By disrupting these complexes, such as treatment with proteases and/or lipases, the RNA can be freed to be used more conveniently for diagnostic purposes or as a target for therapeutics if desired. Thus, for example, if a particular miRNA is believed to cause deleterious effects, exposure of that RNA for activity by, for example, RNAse may precede the treatment with the liberating enzymes. Similarly, the activity of a desirable RNA may be enhanced by liberating it from its protective shields.

The following examples are provided to illustrate but not to limit the invention.

In these examples, plasma or other fluid was analyzed for the various RNA molecules present. Their levels or concentrations in the fluid were also determined.

When plasma was used as a test substrate from human subjects, samples were obtained from Proteogenex (Culver City, Calif.). All samples were collected from donors with proper approvals from institutional review boards. The plasma was prepared from EDTA blood by centrifugation at 1000×g for 15 minutes to separate the plasma and blood cells.

For plasma samples generally, or for cases wherein the sample was a finger-prick of whole blood, total RNA was extracted from 100 μl of the sample using the miRNeasy® kit (Qiagen, Valencia, Calif.). The quality and quantity of the RNA were evaluated with Agilent 2100 Bioanalyzer (Santa Clara, Calif.) and NanoDrop 1000 spectrophotometer (Thermo Scientific, Wilmington, Del.). Generally, we obtained about 100 ng of RNA per ml of sample. As a control we also obtained total RNA from Ambion (Life Technologies, Carlsbad, Calif.).

Libraries of RNA to be sequenced were prepared with small RNA sample preparation kits from Illumina (Illumina, San Diego, Calif.). The 3′ and 5′ adapters, and the reverse transcription primer were diluted in nuclease-free water to the concentration specified by Illumina. RNA isolated from 200 μl of plasma was concentrated and mixed with the diluted 3′ adapter in a final volume of 6 μl of nuclease free water. To eliminate secondary structures, the tube was incubated at 70° C. for 2 minutes, then immediately cooled on ice. The ligation reaction was set by adding 1 μl of 10×T4 RNL2 reaction buffer, 0.8 μl of 100 mM MgCl₂, 1.5 μl of T4 RNA ligase 2, and 0.5 μl of RNaseOUTT™ RNase inhibitor (Life Technologies, Carlsbad, Calif.) and then incubated at 22° C. for 1 hour. After ligating the 3′ adapter, 1 μl of the 5′ adapter, 1 μl of 10 mM ATP, and 1 μl of T4 RNA ligase were added, then incubated at 20° C. for 4 hours.

For cDNA synthesis, 4 μl of RNA ligated with both 5′ and 3′ adapters was mixed with 1 μl of diluted reverse transcription primer and incubated at 70° C. for 2 minutes, then cooled on ice. Two μl of 5× first-strand synthesis buffer, 0.5 μl of 12.5 mM dNTP mix, 1 μl of 100 mM DTT, and 0.5 μl of RNaseOUTT™ were added to the annealed primer-template mixture. The sample was then heated at 48° C. for 3 minutes. One μl of SuperScript II Reverse Transcriptase was added to the sample and incubated at 44° C. for 1 hour. The first-strand cDNA was then amplified with GX1 and GX2 primers using a condition as following: 98° C. for 30 seconds, followed by 20 cycles of 10 seconds at 98° C., 30 seconds at 60° C., 15 seconds at 72° C., holding for 10 minutes at 72° C., then holding at 4° C.

Since the amount of RNA in the sample is low, we did not use the small RNA-enriched fraction for sequencing library preparation; rather we selected and purified through 6% Novex® TBE PAGE gel (Life Technologies, Carlsbad, Calif.) a larger library insert size, covering 20 to 100 nucleotides in length. We thus expected to get lower percentage of sequence reads for miRNA, but would gain the ability to see the general spectrum of RNA in samples including other ncRNAs including bacterial small RNAs (50-500 nt) and degraded messenger RNAs (mRNA).

The quality and quantity of the library was assessed by using the Agilent 2100 Bioanalyzer with the DNA 1000 chip. The prepared library was then run on Illumina Genome Analyzer IIx at the genomic facility at the Institute for Systems Biology.

### Example 1

### Number of RNA Molecules Retrieved and Read

Based on results from 9 individual human subjects, over 20 million sequence reads per sample were obtained with 35 cycle runs on Illumina Genome Analyzer Ia. After trimming the adapter sequences, removing low quality sequences, adapter only sequences, and sequences containing only polyA, we generally had 2 to 4 million “processed” reads with an average length of 23 nucleotides. These data are shown in Table 1.

TABLE 1
Sample Information

| Sample ID | Gender | Age | Ethnic background | Classification | Disease stage | Number of reads | Processed reads |
|---|---|---|---|---|---|---|---|
| D3340P | M | 58 | Caucasian | Normal | | 27,002,901 | 5,085,400 |
| D3176P | F | 52 | Caucasian | Normal | | 27,957,185 | 4,933,712 |
| D3142P | F | 54 | Caucasian | Normal | | 28,214,261 | 4,826,682 |
| 022273P | F | 52 | Caucasian | Colorectal cancer | I | 21,132,674 | 2,183,460 |
| 022163P | F | 56 | Caucasian | Colorectal cancer | III | 23,547,368 | 2,879,950 |
| 022299P | M | 46 | Caucasian | Colorectal cancer | III | 27,957,185 | 4,933,712 |
| 93163P | M | 67 | Caucasian | Ulcerative colitis | 10 ^{(a)} | 20,626,993 | 2,638,989 |
| 93164P | M | 58 | Caucasian | Ulcerative colitis | 11 ^{(a)} | 18,186,259 | 2,807,184 |
| 93166P | F | 48 | Caucasian | Ulcerative colitis | 11 ^{(a)} | 28,214,261 | 4,826,682 |

^{(a)} Based on Mayo Scoring System for Assessment of Ulcerative Colitis Activity

As noted, the total number of reads is greatly diminished by processing as described above which eliminates artifacts due to polyA, adapters, etc.

A NextGen sequence read simulator, ART, available at bioinformatics.joyhz.com/ART/, was used to generate artificial transcriptome data from human, mouse, bovine and yeast. Transcript sequences from ENSEMBL and miRNA sequences from miRBase were combined and used as reference sequences. Illumina read error profile was selected as the program to generate artificial reads with either 23 or 35 nucleotides in length, from the reference sequences. With a 2 mismatch allowance, over 98% of the sequences from our simulated dataset can be mapped to the corresponding transcriptome (Table 3). This provided some assurance that our protocol can map most (˜98%) of the NextGen sequencing data under 2 mismatch allowance.

The nature of the RNA could thus be ascertained. The results for the 9 subjects shown in Table 1 are shown in Table 2 and the results for other species as well as human are shown in Table 3.

TABLE 2
Sequence distribution under different search criteria

| | 0 mismatch | | | 1 mismatch | | | 2 mismatch | | |
|---|---|---|---|---|---|---|---|---|---|
| | Normal | Colorectal | Ulcerative | Normal | Colorectal | Ulcerative | Normal | Colorectal | Ulcerative |
| Category | (3) ^{b} | Cancer (3) ^{b} | colitis (3) ^{b} | (3) ^{b} | Cancer (3) ^{b} | colitis (3) ^{b} | (3) ^{b} | Cancer (3) ^{b} | colitis (3) ^{b} |
| Human microRNA ^{a} | 1.43% | 1.36% | 0.86% | 1.43% | 1.36% | 0.86% | 1.43% | 1.36% | 0.86% |
| Human transcript | 2.78% | 2.73% | 2.44% | 19.27% | 18.68% | 18.52% | 42.30% | 41.07% | 41.82% |
| Human genome | 8.42% | 8.03% | 8.06% | 19.24% | 18.41% | 18.93% | 14.98% | 14.50% | 14.83% |
| Unmapped sequence | 87.38% | 87.87% | 88.64% | 60.06% | 61.55% | 61.69% | 41.29% | 43.06% | 42.49% |

^{a} Due to high sequence similarity for various miRNA species, we did not allow any sequence mismatch in miRNA alignment.
^{b} Numbers in parentheses represents number of samples in each group.

TABLE 3
Sequence distribution using simulated RNA_seq data from different species

| | | Sample | | | | | | | | | | | |
|---|---|---|---|---|---|---|---|---|---|---|---|---|---|
| | Sequence | Human | | | Mouse | | | Bovine | | | Yeast | | |
| Category | length (nt) | 0 MM | 1 MM | 2 MM | 0 MM | 1 MM | 2 MM | 0 MM | 1 MM | 2 MM | 0 MM | 1 MM | 2 MM |
| Endogenous miRNA | 23 | 0.05 | 0.05 | 0.05 | 0.07 | 0.07 | 0.07 | 0.04 | 0.04 | 0.04 | | | |
| Endogenous transcript | 23 | 87.93 | 99.23 | 99.89 | 87.88 | 99.21 | 99.89 | 87.84 | 99.18 | 99.85 | 87.82 | 99.27 | 99.98 |
| Endogenous genome | 23 | 0.12 | 0.02 | 0.01 | 0.08 | 0.02 | 0.01 | 0.07 | 0.02 | 0.01 | 0.00 | 0.00 | 0.00 |
| Unmapped Sequence | 23 | 0.31 | 0.00 | 0.00 | 0.17 | 0.00 | 0.00 | 0.31 | 0.02 | 0.01 | 0.09 | 0.00 | 0.00 |
| Endogenous miRNA | 35 | 0.02 | 0.02 | 0.02 | 0.01 | 0.01 | 0.01 | 0.03 | 0.03 | 0.03 | | | |
| Endogenous transcript | 35 | 60.90 | 91.54 | 98.75 | 60.92 | 91.56 | 98.76 | 60.88 | 88.95 | 96.02 | 60.74 | 91.36 | 98.72 |
| Endogenous genome | 35 | 0.17 | 0.05 | 0.01 | 0.09 | 0.03 | 0.01 | 0.09 | 0.03 | 0.01 | 0.00 | 0.00 | 0.00 |
| Unmapped Sequence | 35 | 38.91 | 8.39 | 1.22 | 38.98 | 8.40 | 1.22 | 39.00 | 10.99 | 3.94 | 39.26 | 8.64 | 1.28 |

MM is abbreviation for “mismatch”, shown in percentages.

The processed sequences were first screened against endogenous (human) sequence databases including known human miRNA, human transcripts, followed by human genomic sequence. To get complementary and efficient mapping results, the alignment tool BLAST was used to search miRNA, and Bowtie was used to search other large databases. For the endogenous sequence mapping, except miRNA, we applied three different levels of error tolerance: 0 mismatch (termed Strategy 0), 1 mismatch (termed Strategy 1) and 2 mismatch (termed Strategy 2). The remaining unmapped sequences were then compared to sequences from the known human microbiome, miRNA sequences from other species, and the non-redundant nucleic acid sequence collection from NCBI. Due to the high sequence similarity for miRNA, we did not allow any sequence mismatch for either endogenous and exogenous miRNA mappings. We also did not allow any sequence mismatches for exogenous sequence mapping. Species classification was based on NCBI Taxonomy database at ncbi nlm nih.gov/taxonomy.

As shown in Table 2 for the 9 human subjects, a large portion of the RNA could not be matched to the database although this percentage diminished as less rigorous requirements for matching were employed as in strategy allowing for two mismatches.

On first examination, we noticed that less than 1.5% of the processed reads actually mapped to human miRNAs. About 11% of the remaining reads mapped to human transcripts and human genome sequence when no sequence mismatch was allowed (Table 2). With a higher tolerance of sequence mismatches, the fraction of reads that can be mapped rose to about 42% to known human transcripts and 15% to other human genomic sequences (under two mismatch allowance). However, this still leaves over 40% of the processed reads with an unknown origin.

### Example 2

### The Presence of Exogenous RNA in Human Plasma

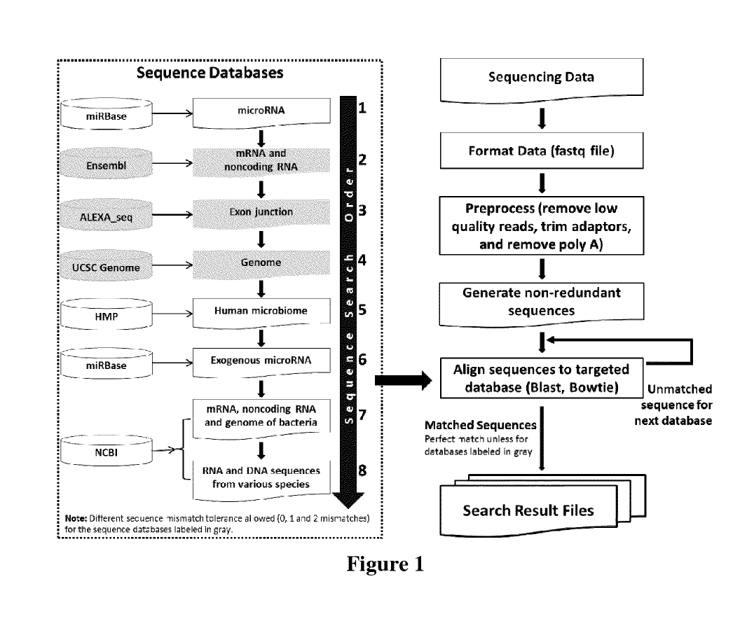

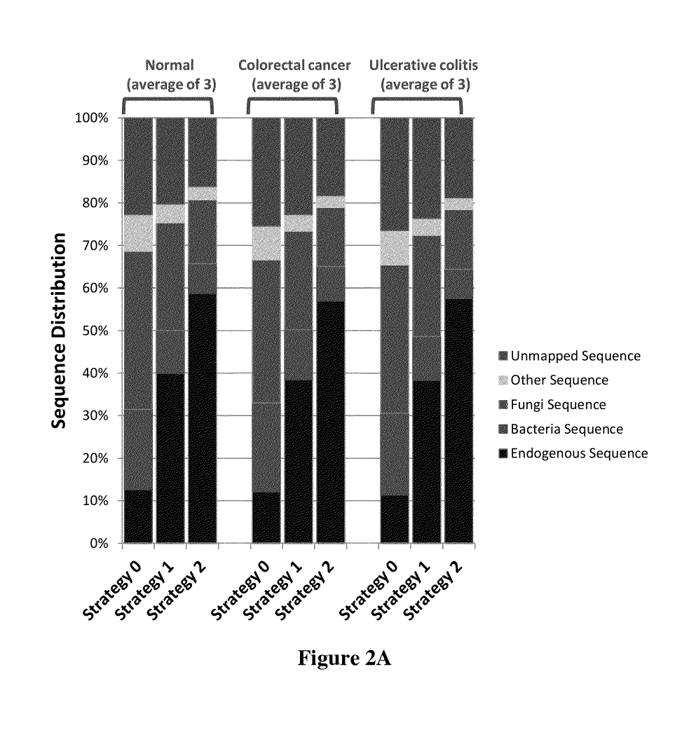

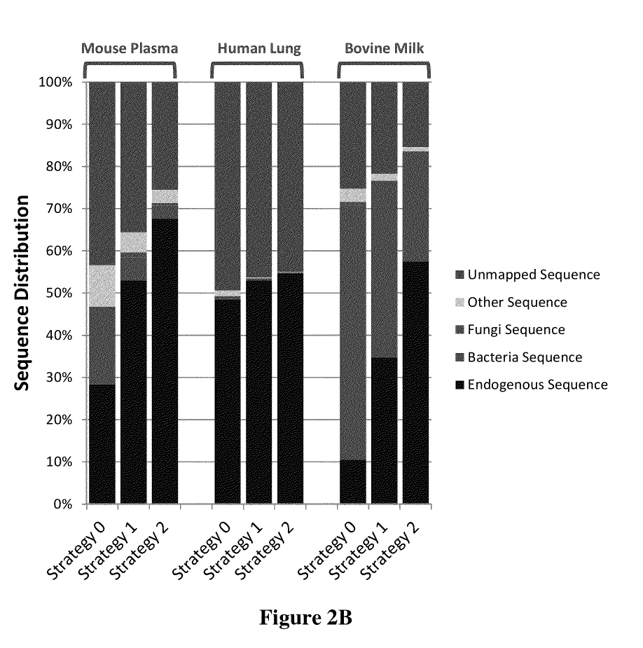

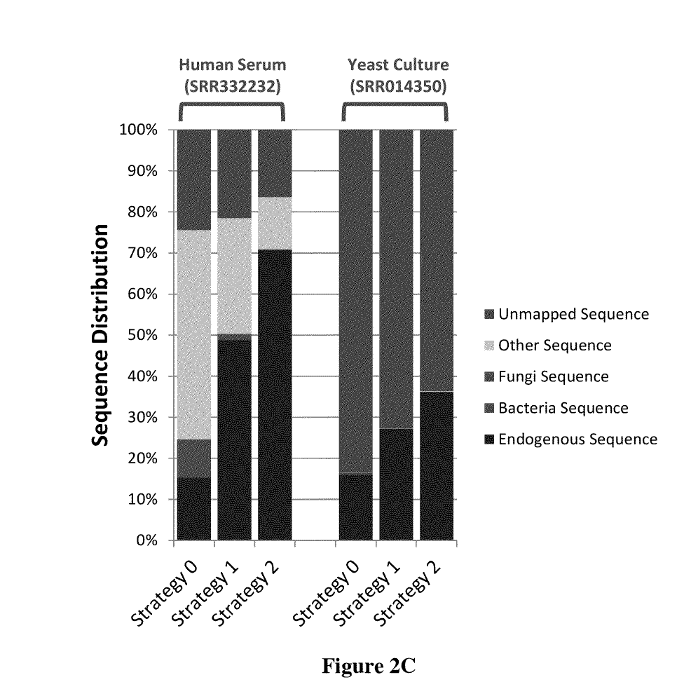

In order to identify the origin of those unmapped sequences in our sequencing results and to ensure that there was no error introduced in preparing the sequencing library that could account for the unknowns, we conducted a systematic search against various sequence databases. We used a “map and remove” approach to analyze the sequence as shown in FIG. 1. The processed sequences were first screened against endogenous (human) sequence databases including known human miRNA, human transcripts, followed by human genomic sequence. Except for the miRNA (since some of the miRNAs have very similar sequences), we applied three different levels of error tolerance, 0 mismatch (termed Strategy 0), 1 mismatch (termed Strategy 1) and 2 mismatches (termed Strategy 2) for the endogenous sequence mapping. The remaining unmapped sequences were then compared to sequences from the known human microbiome, miRNA sequences from other species, and the non-redundant nucleic acid sequence collection from NCBI without any mismatch allowance. To our surprise, a significant number of the unmapped reads aligned with various bacterial and fungal sequences as shown in FIG. 2A and Table 4.

TABLE 4
Sequence Distribution Under Different Search Criteria for Human Plasma Samples

| | Sample | | | | | | | | |
|---|---|---|---|---|---|---|---|---|---|
| | Normal (3) ^{a} | | | Colorectal cancer (3) ^{a} | | | Ulcerative colitis (3) ^{a} | | |
| Search method | Strategy 0 | Strategy 1 | Strategy 2 | Strategy 0 | Strategy 1 | Strategy 2 | Strategy 0 | Strategy 1 | Strategy 2 |
| Endogenous Sequence | 12.62% | 39.94% | 58.71% | 12.13% | 38.45% | 56.94% | 11.36% | 38.31% | 57.51% |
| Bacterial Sequence ^{b} | 18.83% | 10.10% | 7.03% | 20.86% | 11.73% | 8.08% | 19.20% | 10.25% | 6.87% |
| Fungal Sequence ^{b} | 37.20% | 25.32% | 14.98% | 33.64% | 23.17% | 13.90% | 34.85% | 23.84% | 14.06% |
| Other Sequence ^{b} | 8.55% | 4.30% | 3.06% | 7.93% | 3.85% | 2.71% | 8.09% | 3.92% | 2.71% |
| Unmapped Sequence | 22.80% | 20.34% | 16.22% | 25.45% | 22.80% | 18.37% | 26.50% | 23.68% | 18.85% |

^{a} Numbers in parentheses represent number of samples in each group.
^{b} To increase the sequence mapping accuracy, we did not allow any sequence mismatch except in the endogenous sequence search step.

### Example 3

### Exogenous RNA in Other Species and Sample Types

To eliminate the possibility of bacteria and fungi contamination during plasma preparation and handling, we generated sequencing libraries from other types of samples including human tissue (commercially obtained normal lung RNA), bovine milk (commercial whole milk), and mouse plasma (C57BL/6J), and proceeded through the same analysis scheme. Sequences from bacteria, fungi and other species can also be seen in these samples (FIG. 2B and Table 5).

TABLE 5
Sequence Distribution Under Different Search Criteria for Different Types of Samples

| | Sample | | | | | | | | |
|---|---|---|---|---|---|---|---|---|---|
| | Mouse Plasma (2) ^{a} | | | Human Lung | | | Bovine Milk | | |
| Search method | Strategy 0 | Strategy 1 | Strategy 2 | Strategy 0 | Strategy 1 | Strategy 2 | Strategy 0 | Strategy 1 | Strategy 2 |
| Endogenous Sequence | 28.37% | 53.02% | 67.59% | 48.54% | 53.05% | 54.62% | 10.52% | 34.78% | 57.51% |
| Bacterial Sequence ^{b} | 11.34% | 5.40% | 3.10% | 0.15% | 0.06% | 0.05% | 60.43% | 41.72% | 26.04% |
| Fungal Sequence ^{b} | 7.05% | 1.21% | 0.63% | 0.56% | 0.41% | 0.29% | 0.67% | 0.19% | 0.06% |
| Other Sequence ^{b} | 9.85% | 4.73% | 3.15% | 1.37% | 0.21% | 0.08% | 3.09% | 1.57% | 1.01% |
| Unmapped Sequence | 43.39% | 35.64% | 25.53% | 49.38% | 46.27% | 44.96% | 25.29% | 21.74% | 15.38% |

^{a} Numbers in parentheses represent number of samples in each group.
^{b} To increase the sequence mapping accuracy, we did not allow any sequence mismatch except in the endogenous sequence search step.

The overall percentages of exogenous sequences for mouse plasma were lower compared to human plasma samples. The human lung tissue had a very small fraction: less than 1% under strategies 1 and 2, of the processed sequences were from exogenous sources. The commercially obtained milk contains a significant fraction of sequences mapped to bacteria.

To ensure that the exogenous sequences we observed were not derived from any contaminated instruments or reagents, we analyzed two public domain NextGen sequencing data sets: SRR332232, serum small RNA sequencing results from a normal Chinese individual, and SRR014350, yeast transcriptome data from a yeast culture. The yeast culture should not have any exogenous sequences since it was grown in a sterile, defined culture media. The yeast dataset yielded less than 0.15% of the reads mapped to sequences other than yeast (FIG. 2C and Table 6), a level that is fully attributable to coincidence caused by sequencing errors. Using our sequencing analysis pipeline, by contrast, we observed that about 12% of the sequences in human serum sample were from various exogenous species under Strategy 2.

TABLE 6
Sequence Distribution under Different Search Criteria for Two Public Domain Sequences

| | Sample | | | | | |
|---|---|---|---|---|---|---|
| | Human Serum (SRR332232) ^{a} | | | Yeast (SRR014350) ^{a} | | |
| Search Method | Strategy 0 | Strategy 1 | Strategy 2 | Strategy 0 | Strategy 1 | Strategy 2 |
| Endogenous Sequence | 15.33% | 48.75% | 70.78% | 16.01% | 27.16% | 36.16% |
| Bacteria Sequence ^{b} | 7.79% | 1.09% | 0.04% | 0.28% | 0.02% | 0.00% |
| Fungi Sequence ^{b} | 1.47% | 0.46% | 0.08% | 0.11% | 0.11% | 0.11% |
| Other Sequence ^{b} | 50.96% | 28.14% | 12.66% | 0.09% | 0.03% | 0.03% |
| Unmapped Sequence | 24.45% | 21.56% | 16.44% | 83.51% | 72.68% | 63.70% |

^{a} Numbers in parentheses are the access numbers.
^{b} To increase the sequence mapping accuracy, we did not allow any sequence mismatch except in the endogenous sequence search step.

### Example 4

### Human Microbiomes

As noted in Example 2, allowing 2 mismatches identifies 98% of the endogenous sequences in humans. The exogenous sequence mapping results from Strategy 2 (2 mismatches allowed for endogenous sequence mapping steps and no mismatch allowed in exogenous sequence mapping) was used for further analysis.

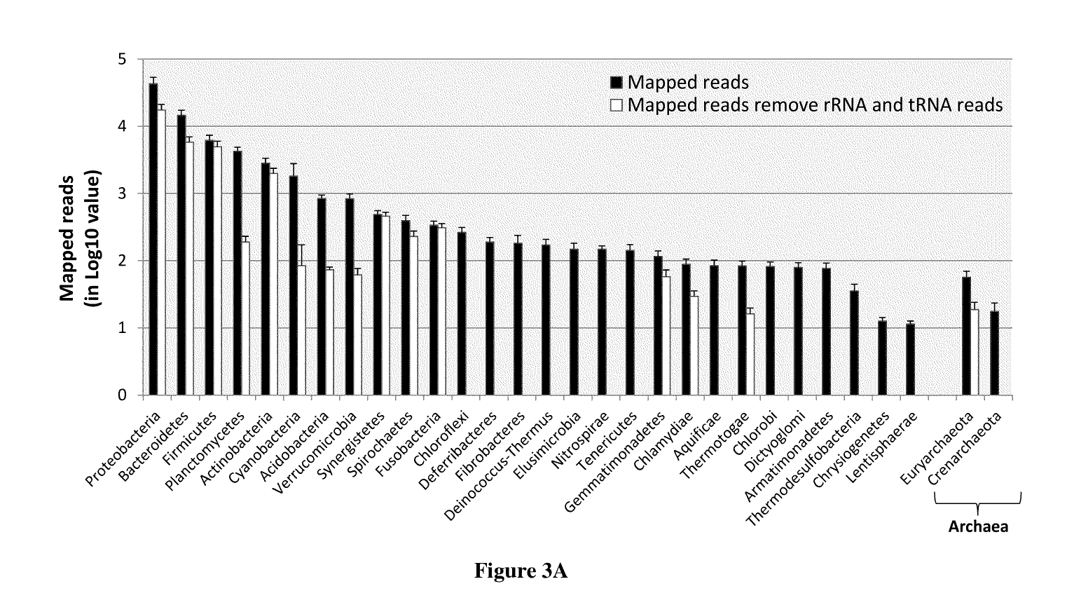

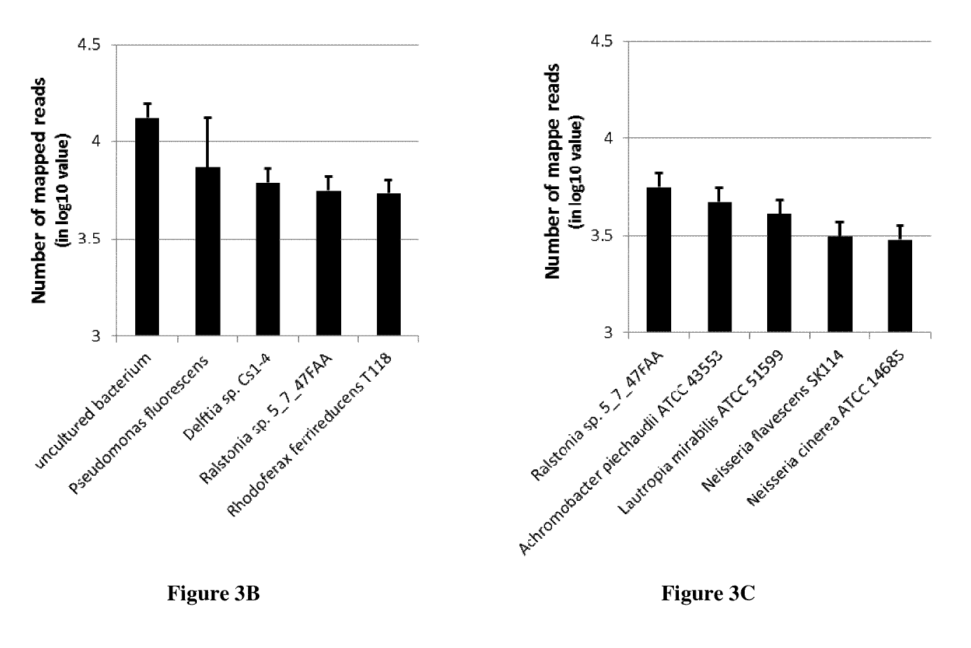

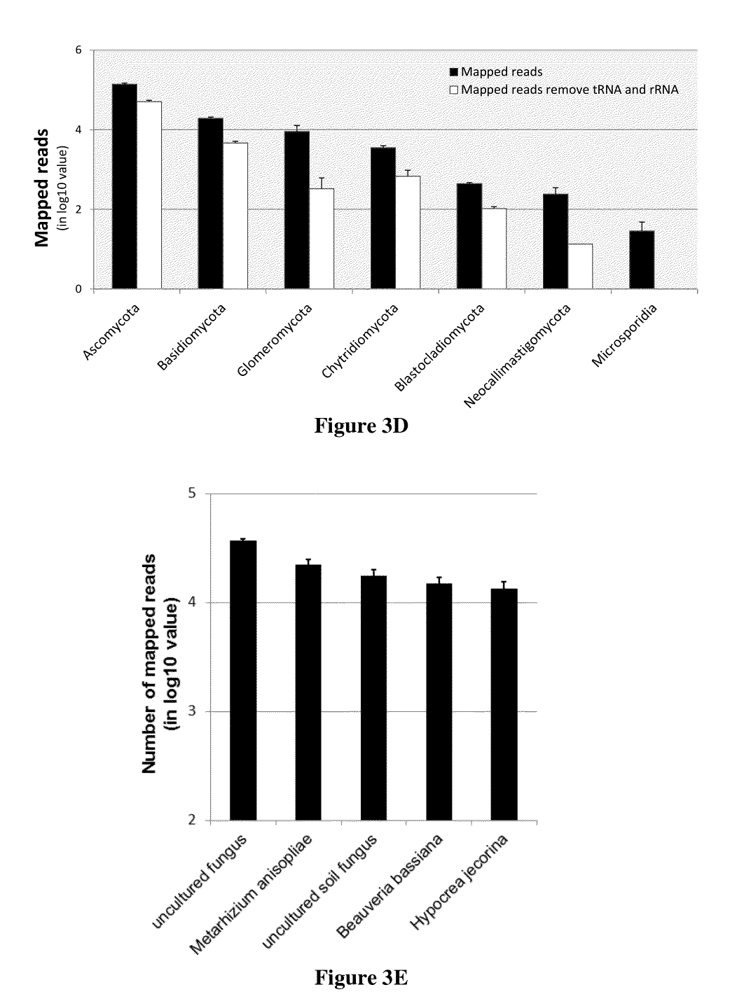

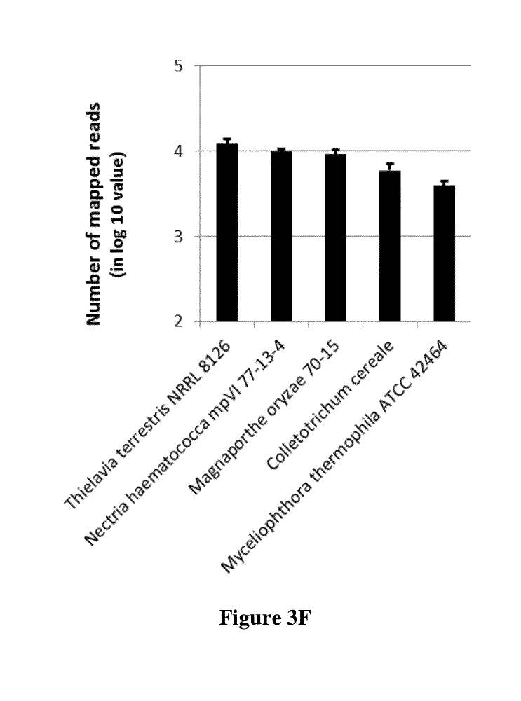

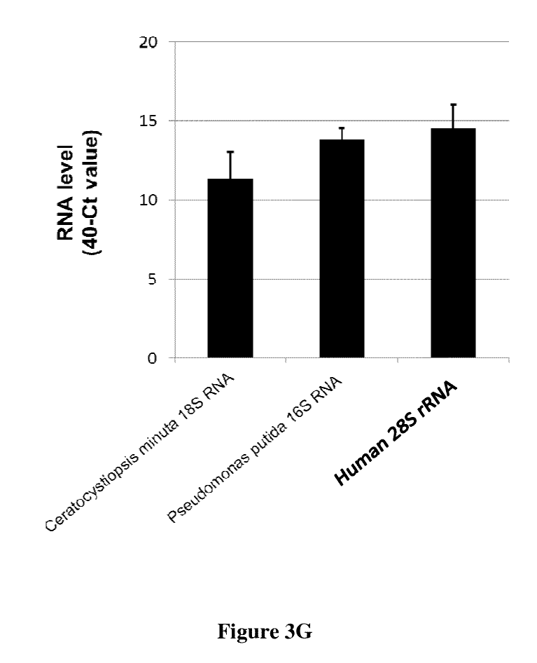

We observed reads from human plasma covering all major bacteria phyla and two archaeal phyla [Euryarchaeota (include methanogens typically found in intestines) and Crenarchaeota] as shown in FIG. 3A and Table 7.

TABLE 7
Distribution of Exogenous Sequences Mapped to Human
Plasma Microbiome Based on Kingdom and Phylum

| | | | | | Mapped sequence remove | | |
|---|---|---|---|---|---|---|---|
| | | Mapped sequence (log 10) | | | tRNA and rRNA (log 10) | | |
| | | | Colorectal | Ulcerative | | Colorectal | Ulcerative |
| Kingdom | Phylum | Normal | cancer | colitis | Normal | cancer | colitis |
| Bacteria | Proteobacteria | 4.62 | 4.69 | 4.59 | 4.22 | 4.29 | 4.22 |
| | Bacteroidetes | 4.14 | 4.18 | 4.17 | 3.74 | 3.79 | 3.77 |
| | Firmicutes | 3.76 | 3.84 | 3.77 | 3.66 | 3.75 | 3.68 |
| | Planctomycetes | 3.63 | 3.63 | 3.62 | 2.29 | 2.28 | 2.26 |
| | Actinobacteria | 3.42 | 3.49 | 3.44 | 3.27 | 3.34 | 3.28 |
| | Cyanobacteria | 3.18 | 3.37 | 3.23 | 1.81 | 2.07 | 1.9 |
| | Acidobacteria | 2.92 | 2.94 | 2.93 | 1.87 | 1.89 | 1.84 |
| | Verrucomicrobia | 2.91 | 2.94 | 2.92 | 1.76 | 1.81 | 1.78 |
| | Synergistetes | 2.67 | 2.71 | 2.69 | 2.64 | 2.68 | 2.66 |
| | Spirochaetes | 2.55 | 2.62 | 2.63 | 2.31 | 2.38 | 2.39 |
| | Fusobacteria | 2.51 | 2.55 | 2.52 | 2.48 | 2.51 | 2.48 |
| | Chloroflexi | 2.37 | 2.45 | 2.44 | | | |
| | Deferribacteres | 2.23 | 2.31 | 2.3 | | | |
| | Fibrobacteres | 2.18 | 2.27 | 2.33 | | | |
| | Deinococcus- | 2.18 | 2.27 | 2.26 | | | |
| | Thermus | | | | | | |
| | Elusimicrobia | 2.15 | 2.21 | 2.16 | | | |
| | Nitrospirae | 2.16 | 2.18 | 2.17 | | | |
| | Tenericutes | 2.11 | 2.2 | 2.15 | | | |
| | Gemmatimonadetes | 2.01 | 2.09 | 2.1 | 1.69 | 1.79 | 1.8 |
| | Chlamydiae | 1.9 | 1.95 | 1.99 | 1.42 | 1.47 | 1.53 |
| | Aquificae | 1.88 | 1.95 | 1.95 | | | |
| | Thermotogae | 1.87 | 1.95 | 1.95 | 1.16 | 1.21 | 1.26 |
| | Chlorobi | 1.86 | 1.94 | 1.94 | | | |
| | Dictyoglomi | 1.87 | 1.92 | 1.91 | | | |
| | Armatimonadetes | 1.87 | 1.88 | 1.9 | | | |
| (Bacteria) | Thermodesulfobacteria | 1.48 | 1.6 | 1.58 | | | |
| | Chrysiogenetes | 1.08 | 1.14 | 1.07 | | | |
| | Lentisphaerae | 1.03 | | 1.07 | | | |
| Archaea | Euryarchaeota | 1.68 | 1.79 | 1.8 | 1.19 | 1.34 | 1.3 |
| | Crenarchaeota | | 1.33 | 1.16 | | | |
| Fungi | Ascomycota | 5.16 | 5.13 | 5.13 | 4.72 | 4.7 | 4.69 |
| | Basidiomycota | 4.3 | 4.27 | 4.29 | 3.68 | 3.65 | 3.67 |
| | Glomeromycota | 3.95 | 3.96 | 3.95 | 2.51 | 2.5 | 2.54 |
| | Chytridiomycota | 3.58 | 3.55 | 3.51 | 2.92 | 2.79 | 2.78 |
| | Blastocladiomycota | 2.57 | 2.72 | 2.64 | 1.87 | 2.26 | 1.92 |
| | Neocallimastigomycota | 2.32 | 2.32 | 2.52 | 1.08 | 1.18 | 1.12 |
| | Microsporidia | 1.57 | 1.4 | 1.41 | | | |

As shown in Table 7, significant difference was observed in the sequence distribution patterns among plasma samples from normals and patients with either colorectal cancer or ulcerative colitis. Firmicutes, typically on of the two most abundant bacteria phyla in the human gut microbiome, is the 3rd most abundant sequence population in plasma.

A significant number of the reads mapped to bacteria are from various ribosomal RNAs and tRNAs. High sequence similarity of these sequences among different microbial species can easily lead to misassignment of sequence reads. Thus, to increase the reliability of mapping results, we removed reads that mapped to bacterial rRNAs and tRNAs and reanalyzed. Removing rRNA and tRNA sequences affected our ability to detect species from Chloroflexi, Deferribacteres, Fibrobacteres and some other phyla (FIG. 3A). The Proteobacteria are still the most abundant phylum followed by Bacteroidetes and Firmicutes.

The bacterium that accounts for the highest number of reads is an uncultured bacterium. This is followed by Pseudomonas fluorescens, an important beneficial bacterium in agricultural settings (FIG. 3B). After removing the tRNA and rRNA reads, a bacterium from Ralstonia becomes the most abundant source followed by Achromobascter piechaudii, a bacterium identified from some clinical samples (FIG. 3C).

Fungi represent the largest source of exogenous RNA, about 14% of the processed reads under the Strategy 2 in our human plasma samples as shown in Example 2 (Table 4). Like bacteria, the species mapped covered all major phyla in fungi and Ascomycota is the most abundant phylum in either with or without rRNA and tRNA reads (FIG. 3D and Table 7 above). No species from Microsporidia were detected after removal of rRNA and tRNA sequences.

Metarhizium anisopliae, a common fungus in soil had the most mapped reads and Thielavia terrestris, a thermophilic fungus became the species with the most abundant reads after removing tRNA and rRNA sequences (FIGS. 3E and 3F). We also observed a significant number of reads mapped to yeast (Saccharomyces cerevisiae) used in baking and brewing either with or without rRNAs and tRNAs. The number of mapped reads was 2-3.5 in log 10 value and did not seem to vary substantially among the 9 subjects.

We recently developed a qPCR based protocol to measure the level of RNA molecules directly from small amount of plasma without RNA isolation (Wang, et al., in preparation). Using this approach we were able to detect both Pseudomonas putida (bacterium) 16S RNA, Ceratocystiopsis minuta (fungus) 18S RNA along with the human 28S rRNA from freshly obtained plasma from finger-prick blood samples (FIG. 3G).

We also compared the data in FIG. 3G with filtered samples to ensure further that no outside contamination was distorting the results. We used the 0.2 μM filter commonly used in tissue culture to eliminate bacteria and fungi contamination, to filter the plasma samples before RNA isolation, and did not observe any significant difference in exogenous RNA levels between filtered and unfiltered plasma, using QPCR primers specific to Pseudomonas putida 16S RNA and Ceratocystiopsis minuta 18S RNA, matching the results for the human 28S rRNA.

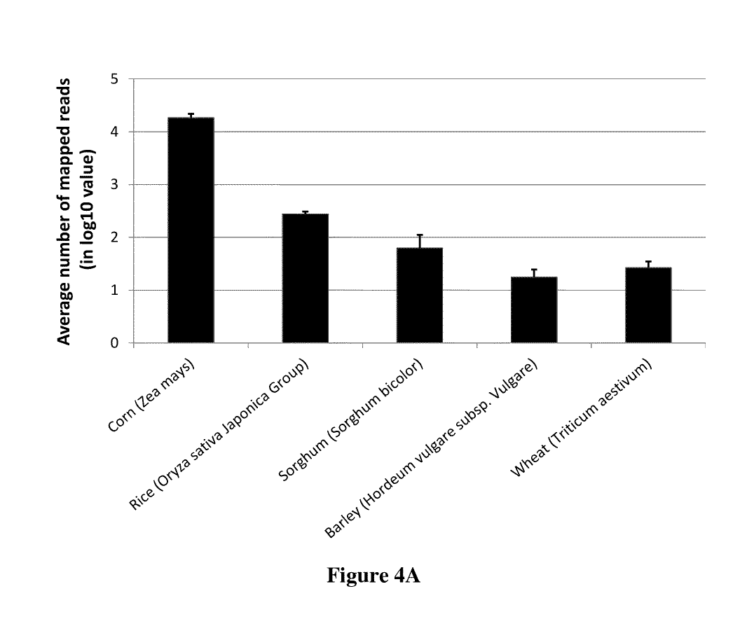

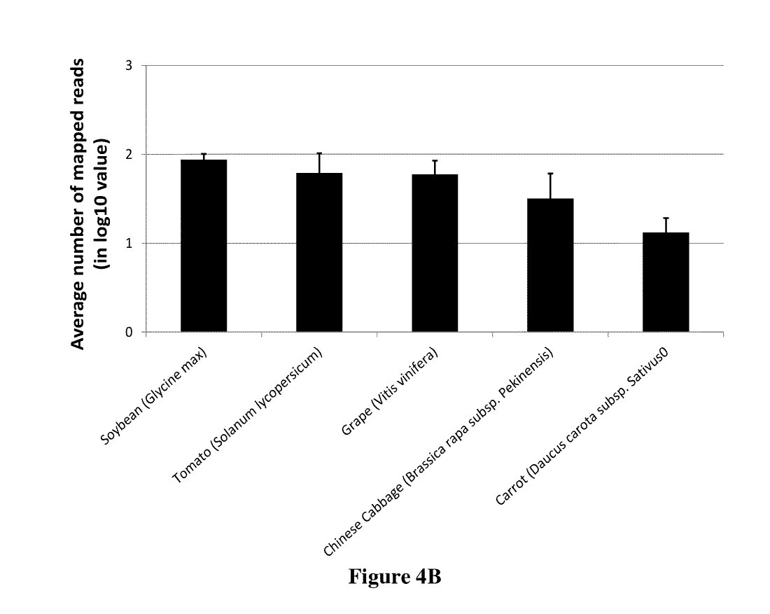

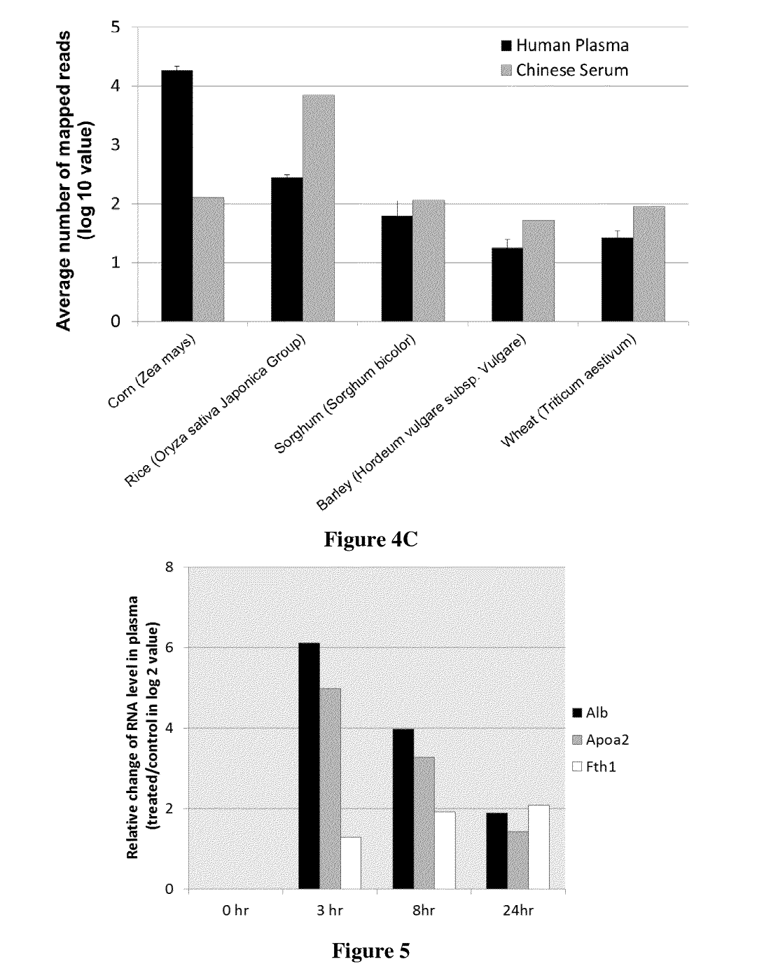

After removing the reads that mapped to rRNAs and tRNAs to increase the accuracy of mapping results, we found reads that mapped to food items. We did not analyze sequences mapped to metazoan species since the risk of coincidental sequence match caused by sequencing error is much higher between human and some metazoan samples. The most abundant food item identified from our plasma samples then is corn (Zea mays) followed by rice (Oryza sativa Japonica Group) (FIG. 4A). The number of mapped reads to corn is 66 times higher on average than rice. In comparing the data from the serum sample from a Chinese individual (downloaded from the public domain: SRR332232), we found that the sequence abundance between corn and rice is reversed: rice has the highest number of reads, by about 55-fold over the number from corn (FIG. 4B). Besides the common cereal grains, we also observed RNA from other food items including soybeans (Glycine max), tomato (Solanum lycopersicum), grape (Vitis vinifera) and others in our plasma samples (FIG. 4C).

### Example 5

### Exogenous miRNAs from Other Species in Human Plasma

Our sequencing results also revealed the presence of exogenous miRNAs from other species. Due to the extreme sequence similarity of miRNA sequences among some species, it is often difficult to determine the exact origin of those exogenous miRNAs. Some of the highly abundant exogenous miRNA species detected in our plasma samples are listed in Table 8.

TABLE 8
List of some abundant exogenous miRNA species identified in plasma

| | Normal | | | Colorectal cancer | | | Ulcerative colitis | | |
|---|---|---|---|---|---|---|---|---|---|
| Sample ID | D3340P | D3176P | D3142P | 022273P | 022163P | 022299P | 93163P | 93164P | 93166P |
| tca-miR-263a-5p | 678 | 3 | 2 | 1 | 1 | 3 | 57 | 2 | 2 |
| nvi-bantam; tca-bantam; dpu-bantam; isc-bantam; | 0 | 0 | 0 | 2 | 1 | 0 | 173 | 3 | 0 |
| ame-bantam | | | | | | | | | |
| zma-miR168a; sbi-miR168; sof-miR168a; osa-miR168a; | 12 | 13 | 10 | 7 | 6 | 13 | 6 | 8 | 10 |
| ssp-miR168a; bdi-miR168; hvu-miR168-5p; zma- | | | | | | | | | |
| miR168b | | | | | | | | | |
| dan-bantam; dwi-bantam; dme-bantam; dps-bantam; | 52 | 1 | 1 | 0 | 0 | 1 | 0 | 0 | 1 |
| dgr-bantam; dya-bantam; aae-bantam; dse-bantam; | | | | | | | | | |
| dmo-bantam; dvi-bantam; dsi-bantam; der-bantam; | | | | | | | | | |
| dpe-bantam | | | | | | | | | |
| dps-miR-8; ame-miR-8; dgr-miR-8; dme-miR-8-3p; | 22 | 0 | 1 | 0 | 0 | 0 | 0 | 1 | 1 |
| cte-miR-8; nvi-miR-8; dwi-miR-8; isc-miR-8; | | | | | | | | | |
| tca-miR-8-3p; dpe-miR-8; nlo-miR-8; der-miR-8; | | | | | | | | | |
| dan-miR-8; lgi-miR-8; bmo-miR-8; aae-miR-8; | | | | | | | | | |
| aga-miR-8; dya-miR-8; dse-miR-8; dvi-miR-8; | | | | | | | | | |
| dsi-miR-8; dpu-miR-8; dmo-miR-8 | | | | | | | | | |
| bma-miR-228 | 0 | 0 | 8 | 0 | 0 | 0 | 0 | 0 | 8 |
| cte-miR-252a; dsi-miR-252; dps-miR-252; sko- | 14 | 0 | 0 | 0 | 0 | 0 | 2 | 0 | 0 |
| miR-252a; nvi-miR-252; dme-miR-252-5p; cqu- | | | | | | | | | |
| miR-252; bmo-miR-252 | | | | | | | | | |
| api-miR-263b | 0 | 0 | 0 | 0 | 0 | 0 | 10 | 0 | 0 |
| dpu-mir-263a; aae-mir-263a; cqu-mir-263; | 9 | 0 | 0 | 0 | 0 | 0 | 1 | 0 | 0 |
| bmo-mir-263a | | | | | | | | | |

Except for miR-168a from the common cereal grains such as corn or rice, the rest of the exogenous miRNAs were from various common household insects, including the housefly, mosquito and bees. There is a high variation in the number of reads among individual donors for those insect miRNAs.

### Example 6

### Response of RNA Spectrum to Physiological Stress

An acetaminophen overdose mouse model for drug-induced liver injury was employed to determine the effect of liver injury on the RNA spectrum. Several hundred transcripts in plasma were affected including those representing transcripts that are highly concentrated in liver such as albumin, apolipoprotein A2 (apoA2) and transferrin. All of these were significantly increased as compared to untreated controls. The level of albumin spiked after 3 hours and decreased over a 24 hour period, as did that of apoA2. The transferrin levels were increased to a lesser extent but held reasonably steady over a 24-hour period (FIG. 5).

In addition, we used a gene enrichment analysis from the Database for Annotation, Visualization and Integrated Discovery (DAVID) found on the web at david.abcc.ncifcrf.gov/home/jsp. The enrichment of organ-specific transcripts in blood as well as liver is shown in Table 9.

TABLE 9

| | Increased Level in Plasma | | | Decreased Level in Plasma | | |
|---|---|---|---|---|---|---|
| Tissue | 3 hr | 8 hr | 24 hr | 3 hr | 8 hr | 24 hr |
| Liver | 2.50E−16 | 8.70E−07 | 4.20E−03 | | | 3.70E−02 |
| Bone marrow | 4.90E−04 | 1.20E−04 | 4.40E−04 | | 4.70E−02 | 2.30E−02 |
| Kidney | 6.20E−04 | 2.00E−03 | 9.70E−03 | 3.70E−02 | 3.90E−02 | 2.90E−02 |
| Small intestine | 1.60E−02 | | | | | 7.50E−02 |
| Colon | 4.00E−02 | | | | | |
| Spleen | | 3.50E−02 | 8.30E−02 | | | 6.70E−02 |
| Skeletal muscle | | | | | 9.70E−02 | |
| Stomach | | | | 3.90E−02 | | |
| Lung | | 6.10E−02 | | | | |

The numbers in the table are p values that represent the likelihood of the tissue origin of the RNA sequences observed in plasma, and are smaller the greater the likelihood this is the case. Thus, in the case of liver, the certainty that the increase in transcripts from liver was most certain at 3 hours and less so at 24 hours. As shown in Table 9, some transcripts derived from liver increased significantly in plasma post-acetaminophen administration which suggests RNA released from hepatocyte due to acetaminophen induced liver injury. Histopathology examination of the liver tissues indicates typical zoon 3 hepatocyte death induced by acetaminophen overdose. The other major organ listed in Table 9 is kidney. Histopathological examination on the kidney tissues also indicates renal tubular injury induced by acetaminophen overdose. These findings provide the evidences of using the spectrum of endogenous RNA to predict pathology occurred in specific tissues.

The effect of a particular condition, asymptomatic sarcoidosis, a systemic inflammatory disease with granulomas in multiple tissues also provided a pattern of transcripts detectable in plasma characteristic of various organs as shown in Table 10.

TABLE 10
Asymptomatic Sarcoidosis

| Organ | Increase in plasma | Decrease in plasma |
|---|---|---|
| Endometrium | 9.40E−04 | |
| Peripheral Nervous System | 2.80E−03 | |
| Brain | 2.90E−03 | 6.00E−06 |
| Epithelium | 1.80E−02 | 2.10E−02 |
| Lung | 2.00E−02 | |
| Bone marrow | 2.50E−02 | |
| Pancreas | 2.70E−02 | |
| Retina | 3.60E−02 | |
| Platelet | 5.00E−02 | |
| Kidney | | 5.70E−03 |
| Skin | | 3.50E−02 |
| Muscle | | 7.80E−02 |

### Example 7

### Stability of RNA in Plasma

To explore the stability of exogenous RNA in plasma, we treated the plasma with RNase A from Fermentas™ (Thermo Scientific™, Wilmington, Del.) at a concentration at 1 μg/ml, DNase I (Fermentas™, Thermo Scientific™, Wilmington, Del.) at a concentration of 1 unit/ml, protease K (Fermentas™, Thermo Scientific™, Wilmington, Del.) at a concentration of 0.05 mg/ml, 0.1% Triton™ X 100, or protease K for 20 minutes followed by additional RNase A at 1 μg/ml after heat inactivation of protease K at 70° C. for 10 minutes prior for RNA isolation.

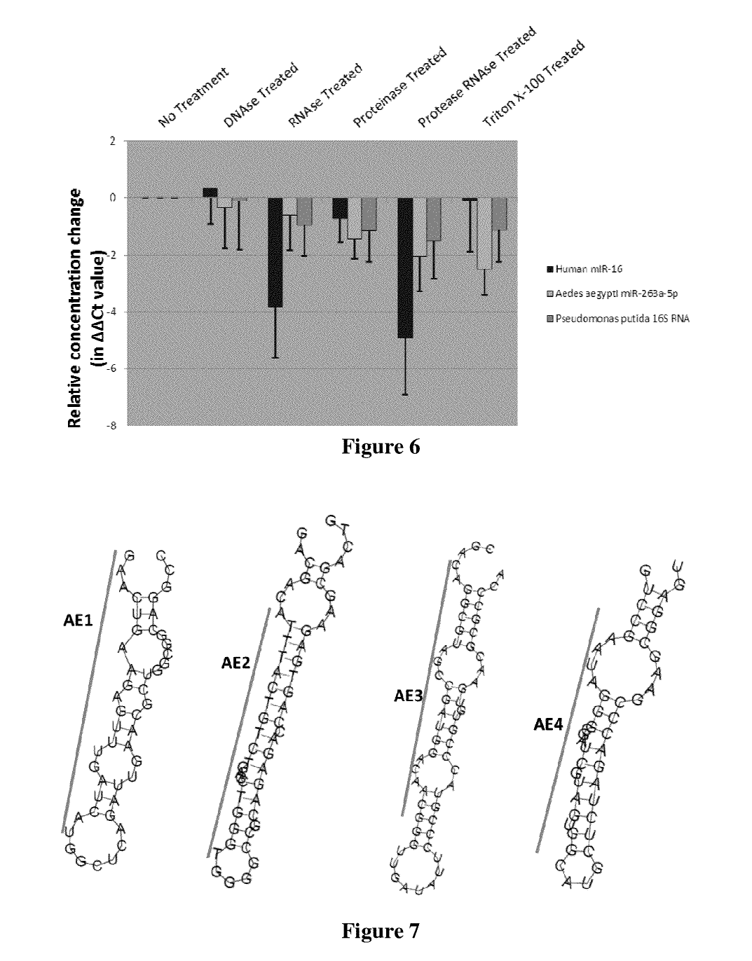

Like endogenous miRNA (miR-16), the levels of specific exogenous miRNA (miR-263a-5p) and RNA (16S rRNA from Pseudomonas putida) were reduced significantly after Triton™ X-100, protease, RNase, and protease followed by RNase treatments (FIG. 6). Adding additional RNase caused less reduction compared to protease followed by RNase treatments. This suggests that some of the exogenous RNA molecules, like endogenous miRNAs, are associated with protein and/or lipid complexes in circulation and a fraction of those complexes may not be tightly bound, such that the freeze thawing process or incubation at 37° C. during enzyme treatment may release some of the protected RNAs.

### Example 8

### Exogenous RNA in Plasma can Affect Cellular Gene Expression Pattern

It has been demonstrated that certain cells can take up miRNA contained in lipid vesicles, resulting in a changed gene expression profile. We transfected several synthetic, double-stranded RNA molecules selected from observed exogenous miRNA sequences and some highly abundant exogenous sequences (bacterial rRNAs) that have potential to form pre-miRNA-like secondary structures (FIG. 7) into a mouse fibroblast cell line.

The mouse dicer deficient (DCR −/−) fibroblast cell line was generated from a conditional cre and floxed Dicer allele transgenic mouse available from Jax (located on the web at jaxmice.jax.org/strain/006001.html) kindly provided by Dr. Jacques Peschon. Part of the RNase III domain encoded in the exon 23 of dicer gene was deleted following cre excision. DCR −/− cells were maintained in Dulbecco's modified Eagle's medium with high glucose. The media was supplemented with 10% FBS, 1% non-essential amino acid, 1% GlutaMAX™. The cells were routinely incubated at 37° C. in a humidified atmosphere with 5% CO₂.

Lipofectamine™ RNAiMAX was purchased from Invitrogen (Life Technologies, Carlsbad, Calif.). Custom designed exogenous RNA used in transfection was obtained from Ambion (Life Technologies, Carlsbad, Calif.). DCR −/− cells were seeded at a density of 1×10⁵ cells in 6-well tissue culture plates 24 h prior to transfect with 10 nM of synthetic RNAs. Cells exposed to transfection reagents only were used as control. After 24 hours in the transfection media, the cells were harvested for RNA isolation and the transfection efficiency was validated with qPCR.

Effects of exogenous RNAs on transcriptome were assessed by using the Agilent mouse 4×44K microarray (Agilent, Santa Clara, Calif.). Total RNAs were isolated with an miRNeasy® column (Qiagen, Valencia, Calif.), and both Cy3 and Cy5-labeled cRNA samples were prepared with two color labeling kit (Agilent Technologies, Santa Clara, Calif.) and then hybridized at 65° C. for 17 h. Signal intensity was calculated from digitized images captured by a scanner from Agilent (Santa Clara, Calif.), and data analysis was performed by using GeneSpring GX software (Agilent Technologies, Santa Clara, Calif.).

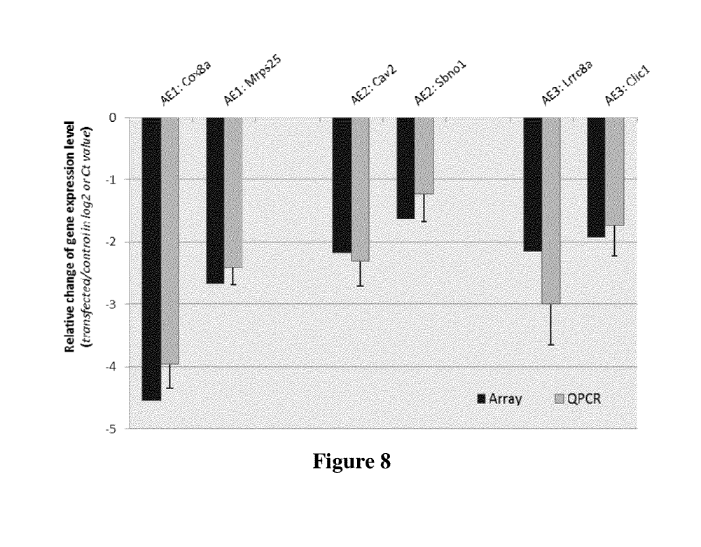

The expression profiles of a number of genes in the cells were affected by some of the exogenous RNA sequences. We verified the changes in levels of some of these affected genes' mRNA by QPCR (FIG. 8). The pathways enriched among those down-regulated genes are listed in Table 11.

TABLE 11
Affected Pathways by Transfecting the Synthetic Exogenous RNA Sequences

| Exogenous RNA | | | |
|---|---|---|---|
| Sequence | Species | Affected pathways | P-Value |
| AE1: GAACUGAAGA | 16S rRNA from | Apoptosis | 3.90E−03 |
| GUUUGAUCAUGG | Pseudomonas | Oocyte meiosis | 1.50E−02 |
| | | Small cell lung cancer | 1.60E−02 |
| | | RNA degradation | 2.00E−02 |
| | | Proteasome | 4.80E−02 |
| | | Pathways in cancer | 5.40E−02 |
| | | Spliceosome | 6.50E−02 |
| | | Pentose phosphate pathway | 7.50E−02 |
| | | Huntington's disease | 9.90E−02 |
| AE2: AUUUACUGUCU | 23S rRNA from | Renal cell carcinoma | 6.70E−03 |
| GAGCUGGGUGG | Rhodococcus | Chronic myeloid leukemia | 8.40E−03 |
| | | Regulation of actin cytoskeleton | 3.10E−02 |
| | | Neurotrophin signaling pathway | 3.50E−02 |
| | | Tight junction | 3.80E−02 |
| | | MAPK signaling pathway | 5.80E−02 |
| | | Chemokine signaling pathway | 8.00E−02 |
| | | ErbB signaling pathway | 8.40E−02 |
| | | Focal adhesion | 9.70E−02 |
| AE3: CAGGCGUAGCC | 23S rRNA from | Pathways in cancer | 1.20E−02 |
| GAUGGACAACG | Rhodococcus | Adipocytokine signaling | 1.50E−02 |
| | | pathway | |
| | | Pancreatic cancer | 1.80E−02 |
| | | Focal adhesion | 1.90E−02 |
| | | Endocytosis | 7.20E−02 |
| | | Cell cycle | 7.80E−02 |
| | | RNA degradation | 8.00E−02 |
| AE4: CGAAUAGGGCG | 23S rRNA from | No enriched pathway | |
| AUCGUAGUGGC | Rhodococcus | | |
| miR-263: AAUGGCAC | miR-263a from | No enriched pathway | |
| UGGAAGAAUUCACGG | mosquito | | |
| Bantam:UGAGAUCA | Bantam from | No enriched pathway | |
| UUGUGAAAGCUGAUU | house fly | | |

Two of the insect miRNAs, miR-263a-5p and bantam, did not produce any significant effects on the cellular transcriptome, which shows that the process of transfection itself was not the cause of the observed gene expression changes. Thus, RNA sequences in plasma have biological effects on human cells.

1. A database contained on a computer readable medium which comprises a record of the identity and levels of RNA sequences or molecules contained in an RNA spectrum associated with at least one of:
1) tissue or biological fluid of normal subjects;
2) tissue or biological fluid of subjects affected by known diseases or conditions;
3) tissue or biological fluid of subjects or administered known treatments;
4) tissue or biological fluid of subjects known to have ingested specified substances.

2. The database of claim 1 wherein said RNA sequences or molecules are 10-40 nucleotides in length.

3. The database of claim 1 wherein the subjects are humans or laboratory animals.

4. The database of claim 1 wherein the sample is biological fluid which is blood, serum or plasma.

5. The database of claim 1 wherein each RNA spectrum includes at least 10 molecules or sequences.

6. The database of claim 1 which includes the RNA spectrum associated with subparagraphs 1) and 2) or
that associated with subparagraphs 1) and 3) or
that associated with subparagraphs 1) and 4).

7. The database of claim 1 wherein the control RNA spectrum is representative of a statistically significant sample of subjects.

8. A method to assess the physiological state of a test subject which method comprises
obtaining a test RNA spectrum in a sample of a tissue or biological fluid from said test subject; and
comparing said RNA spectrum with a control spectrum which is the RNA spectrum of subparagraph 1) of claim 1;
whereby a significant difference between the test spectrum from that of said control spectrum indicates a physiological condition in said test subject that is other than normal.

9. A method to assess the effect of a treatment or protocol that has been administered to a test subject, which method comprises
obtaining a test RNA spectrum in a sample of a tissue or biological fluid from said test subject; and
comparing said spectrum with a control spectrum which is the RNA spectrum of subparagraph 1) of claim 1;
whereby a significant difference between the test spectrum from said control spectrum indicates the effect of said treatment or protocol on said test subject.

10. A method to determine whether a test subject has been subjected to a treatment or protocol or is afflicted with a disease or condition or has ingested a specified substance, which method comprises
obtaining a test RNA spectrum in a sample of a tissue or biological fluid from said subject; and
comparing said spectrum with a control RNA spectrum which is that of subparagraph 2), 3) or 4) of claim 1;
whereby a significant similarity between the test spectrum with that of said control spectrum indicates the subject has been administered said treatment or protocol or is afflicted with said disease or condition or has ingested said substance.

11. The method of claim 8, 9 or 10 wherein the control RNA spectrum is representative of a statistically significant sample of subjects.

12. The method of claim 8, 9 or 10 wherein the test and control subjects are human, or wherein the test and control subjects are laboratory animals.

13. The method of claim 8, 9 or 10 wherein the sample is biological fluid, which is blood, plasma, or serum.

14. A method to determine a microbiome of a test subject, which method comprises the steps of
a) obtaining a test RNA spectrum of RNA sequences or molecules in a sample of a tissue or biological fluid from said test subject; and
b) associating the identity and/or level of RNA molecules in said spectrum with individual microorganisms and/or their function;
whereby the microbiome of said subject is determined.

15. The method of claim 14 wherein RNA sequences or molecules in the RNA spectrum of a) that are endogenous to the subject are deleted from the spectrum before step b) is performed.

16. A method to determine whether a test subject has ingested one or more substances which method comprises the steps of
a) obtaining a test RNA spectrum of RNA sequences or molecules in a sample of a tissue or biological fluid from said subject; and
b) associating the identity and/or level of RNA molecules in said spectrum with said one or more substances;
whereby assessing the presence and/or level of one or more of said RNA molecules as characteristic of said one or more substances determines whether said ingestion has occurred.

17. The method of claim 16 wherein RNA sequences or molecules in the spectrum of a) that are endogenous to the subject are deleted from the spectrum before step b) is performed.

18. A method to expose circulating RNA molecules in the blood, plasma or serum which method comprises treating said blood, plasma or serum with an effective amount of protease and/or lipase.

19. A method to identify a biological pathway that is affected in a subject afflicted with an abnormal condition, which method comprises
identifying at least one RNA molecule in the RNA spectrum of a sample of tissue or biological fluid of said subject, the presence or level of which is different in from that in a control spectrum comparably obtained from control subjects;
testing the effect of said RNA molecule on the transcriptome of cells of the same species as the test subject;
identifying at least one element of said transcriptome that is affected; and
associating said element with a biological pathway.
